(19) United States
(12) Patent Application Publication
(10) Pub. No.: US 2021/0017404 A1
(43) Pub. Date: Jan. 21, 2021

(54) EFFECT PIGMENTS BASED ON COLORED HECTORITES AND COATED COLORED HECTORITES AND MANUFACTURE THEREOF

(71) Applicant: ALTANA AG, Wesel (DE)

(72) Inventors: Michael Grüner, Hartenstein (DE); Günter Kaupp, Hartenstein (DE); Josef Breu, Bayreuth (DE); Josef Hausner, Bayreuth (DE)

(21) Appl. No.: 17/061,988

(22) Filed: Oct. 2, 2020

Related U.S. Application Data
(63) Continuation of application No. PCT/EP2019/058526, filed on Apr. 4, 2019.

(30) Foreign Application Priority Data
Apr. 4, 2018 (EP) 18165577.0

(51) Int. Cl. C09D 5/36; C09D 5/00; C09D 5/03; C09C 1/00; A61K 8/02; C09D 11/037

(52) U.S. Cl. CPC C09D 5/36; C09D 5/002; C09D 5/032; C09C 2200/407; A61K 8/022; C09D 11/037; C09C 2200/1004; C09C 1/0039

(57) ABSTRACT

This invention deals with effect pigment comprising a colored hectorite which is produced by ion exchange process of an initial hectorite with a cationic dye, wherein the initial hectorite can be represented by the formula

K_{z/n}[LiₓMg_{(3.0-(x+y)□y}Si₄O₁₀F₂] (I);
wherein n is the charge of K and z=x+2y with 0.2<z<0.8;
x=0-0.8; y=0-0.4;
K is a cation chosen from a first group consisting of Li⁺, Na⁺, K⁺, NH₄⁺, Rb⁺, Cs⁺, Mg²⁺, Ca²⁺, Ba²⁺ or mixtures thereof or from a second group consisting of alkylammonium salts with 2 to 8 C-atoms, wherein the alkyl can be branched or linear, or from a mixture of cations from the first and the second group and represents not occupied octahedral lattice sites.

Furthermore, these colored hectorites can have a coating thereon comprising at least one layer with a high index of refraction >1.8 or a semitransparent metal and optionally an outer protective layer.

This application is filed as a continuation of International Patent Application No. PCT/EP2019/058526, filed on Apr. 4, 2019, which claims the benefit of EP App. No. 18165577.0, filed on Apr. 4, 2018, which are incorporated herein by reference in their entireties.

This invention is dealing with the coloration of certain layered silicates with cationic dyes, the use thereof as effect pigments and effect pigments, such as pearlescent pigments based on substrates consisting of these colored layered silicates. Furthermore, the invention deals with a method of providing the colored layered silicates and the pearlescent pigments based on these colored layered silicates.

WO 2001/04216 A1 discloses clays which are essentially montmorillonites which can be colored without agglomeration of the clay particles. These colored clays are suitable especially for use in polar polymers.

WO 2001/04050 A1 describes an ionic exchange of a layered inorganic filler, preferably a double-hydroxide, with ionic species which can be cationic dyes. The specific exchange capacity is very low, though.

WO 2000/34379 discloses layered clay intercalated with at least one cationic colorant. The specific exchange capacity is very low, though and the size of the clays is rather low.

WO 1989/09804 discloses clays such as hectorites with high cation exchange capacity layered with cationic dyes leading to a “pigment” without bleeding in water or oil. The particle size is very low, though.

WO 2004/009019 A2: discloses different clays which can be also hectorites with high cation exchange capacity layered with cationic dyes leading to a “pigment” without bleeding in oil. The particle size is very low, though.

WO 2001/0890809 A1 discloses the manufacture of phyllosilicate discs having a high aspect ratio usable for e.g. flame protection barrier of diffusion barrier. These hectorites have an extremely high cationic exchange capacity (CEC). No coloration of these clays is disclosed. The hectorites have to be expected to have a low acid resistance.

WO 2012/175431 A2 discloses large clays with a great degree of delamination, a high layer charge and high aspect ratio. Coloration of these clays is not disclosed therein and is essentially not possible with this type of clays.

M. Stöter, B. Biersack, S. Rosenfeldt, M. J. Leitl, H. Kalo, R. Schobert, H. Yersin, G. O. Ozin, S. Förster and J. Breu, Angew. Chem. Int. Ed. 2015, 54, p. 4963-4967 published a sodium hectorite with high aspect ratio into which a fluorescent dye was intercalated. Without fluorescence this intercalated colored hectorite did not have an optical appealing appearance.

The object of the present invention is to provide clays intercalated with dyes with a high CEC and a high stability against acids. These dye intercalated clays should be usable as effect pigments, i.e. they should have an attractive color impression to the observer and they should be usable as substrates for effect pigments.

A further objective of the present invention is to provide new substrates for the manufacture of effect pigments, especially of pearlescent pigments.

A further objective is to provide cost efficient methods of manufacture of the colored hectorites and of effect pigments based on these materials.

The object of the present invention can be solved by providing an effect pigment comprising a colored hectorite which is produced by ion exchange process of an initial hectorite with a cationic dye, wherein the initial hectorite can be represented by the formula

K_{z/h}[LiₓMg_{(3.0-(x+y)□y}Si₄O₁₀F₂] (I);
wherein n is the charge of K and z=x+2y with 0.2<z<0.8;
x=0-0.8; y=0-0.4;
K is a cation chosen from a first group consisting of Li⁺, Na⁺, K⁺, NH₄⁺, Rb⁺, Cs⁺, Mg²⁺, Ca²⁺, Sr²⁺, Ba²⁺ or mixtures thereof or from a second group consisting of alkylammonium salts with 2 to 8 C-atoms, wherein the alkyl can be branched or linear, or from a mixture of cations from the first and the second group and represents not occupied octahedral lattice sites.

In claims 2 to 11 preferred embodiments of these effect pigments are depicted.

A further object of the present invention can be solved by providing an effect pigment comprising as a substrate a colored hectorite which is produced by ion exchange process of an initial hectorite with a cationic dye, wherein the initial hectorite can be represented by the formula

K_{z/n}[LiₓMg_{(3.0-(x+y)□y}Si₄O₁₀F₂] (I);

wherein n is the charge of K and z=x+2y with 0.2<z<0.8;

x=0-0.8; y=0-0.4;

K is a cation chosen from a first group consisting of Li⁺, Na⁺, K⁺, NH₄⁺, Rb⁺, Cs⁺, Mg²⁺, Ca²⁺, Si²⁺, Ba²⁺ or mixtures thereof or from a second group consisting of alkylammonium salts with 2 to 8 C-atoms, wherein the alkyl can be branched or linear, or from a mixture of cations from the first and the second group and represents not occupied octahedral lattice sites,

and a coating thereon comprising at least one layer with a high index of refraction >1.8 or a semitransparent metal and optionally an outer protective layer.

In claims 13 to 17 preferred embodiments of these effect pigments are depicted.

A further object of the present invention can be solved by providing a method of manufacture of an effect pigment according to claims 1 to 11, comprising the following steps:

a) providing a hectorite according to formula (I)

b) dispersing said hectorite in an aqueous solution or water/acetonitrile or water/alcohol mixtures and optionally impacting of mechanical shear-forces until a highly swollen state is reached by osmotic swelling, wherein the interlayer distance of layers dₛ is in a range of above 10 nm to less than 1,000 nm.

c) ionic exchange of cations K with a cationic dye to a degree of 50-100% of the CEC, wherein optionally a cationic surface modifier is present, wherein the mole ratio of cationic surface modifier to dye is in a range of 0 to 3, and

d) optionally separating the colored hectorites obtained in step b) from the aqueous solution or optionally concentrating the colored hectorites obtained in step b) from the aqueous solution and/or optionally washing the effect pigment.

A further object of the present invention can be solved by providing a method of manufacturing of an effect pigment according to claims 12 to 17, comprising

a) a step of coating an effect pigment of claims 1 to 11 with a high refractive index material, followed by

b) separating or concentrating the coated effect pigment from the solvent of the reaction media of step a),

c) optionally a drying step of the effect pigment of step a) and,

d) optionally classifying the effect pigment.

### DETAILED DESCRIPTION

Effect Pigments Based on Colored Hectorites:

The initial layered silicate used throughout this invention is a 2:1 layered silicate can be represented by the formula (I):

K_{z/n}[LiₓMg_{(3.0-(x+y)□y}Si₄O₁₀F₂] (I);

wherein n is the charge of K and z=x+2y with 0.2<z<0.8; x=0-0.8 and y=0-0.4.

The negative layer charge is denoted to z which is compensated by cations K. K is a cation chosen from a first group consisting of Li⁺, Na⁺, K⁺, NH₄⁺, Rb⁺, Cs⁺, Mg²⁺, Ca²⁺, Sr²⁺, Ba²⁺ or mixtures thereof or from a second group consisting of alkylammonium salts with 2 to 8 C-atoms, wherein the alkyl can be branched or linear, or from a mixture of cations from the first and the second group and represents not occupied octahedral lattice sites.

The 2:1 layered silicates represented by formula (I) are denoted to the class of “hectorites” throughout this invention.

In a first embodiment this invention is directed to effect pigment comprising a colored hectorite which is produced by ion exchange process of an initial hectorite with a cationic dye, wherein the initial hectorite is represented by Formula (I).

In more preferred embodiments the initial hectorite can be represented by the formula (II):

K_{z/n}[LiₓMg_{(3.0-(x+y)□y}Si₄O₁₀F₂]; (II)

wherein n is the charge of K and y=0-0.1 and x=0.35-0.65. The layer charge z is preferably z=0.35 to less than 0.8 and more preferably z=0.4 to 0.7 and most preferably z=0.45 to 0.65.

In this preferred embodiment K is selected from a first group consisting only of the alkali metals K⁺, NH₄₊, Rb⁺, Cs⁺ or mixtures thereof. Preferably K is selected from a group consisting of Li⁺, Na⁺ or mixtures thereof. Most preferably K is selected to be Na⁺ ions.

With cations K such as Li⁺ or Na⁺ hectorites with a rather homogeneous surface layer charge are derivable. Such homogeneous surface charge is a prerequisite, for a strong delamination and thus a strong coloring by cationic dyes.

In another preferred embodiment K is preferably selected from a second group of alkylammonium salts with 2 to 8 C-atoms, wherein the alkyl can be branched or linear.

Preferably these alkylammonium salts are based on alkylamines such as ethylamine, n-propylamine, n-butylamine, sec-butylamine, tert-butylamine, n-pentylamine, tert-amylamine, n-hexylamine, sec-hexylamine, 2-ethyl-1hexylamine, n-heptylamine, 2-aminoheptane, n-octylamine and tert-octylamine or mixtures thereof.

In another embodiments K can be mixtures of the said first and the said second groups.

These hectorites provide a high cationic exchange capacity (CEC) which is preferably in a range of 80 to 213 mval/100 g, more preferably in a range of 100 to 160 mval/100 g and most preferably in a range of 120 to 150 mval/100 g.

A high CEC enables to achieve a high degree of dye adsorption and thus of coloration of the hectorites.

The cationic exchange capacity of such 2:1 layered silicates can be determined by the BaSO₄ method or by the method described by Lagaly (G. Lagaly et al., Clay Miner. 2005, 40, 441-453).

The hectorites chosen according to this invention have the significant advantage that besides their high cationic exchange capacity they exhibit a rather homogeneous surface layer charge which enables them to be delaminated into rather large platelets with respect to the length and width of the platelet-like particles.

This is in contrast to layered silicates such as e.g. montmorillonites where only small particle sizes are feasible.

Effect pigment based on the colored hectorites have a lateral dimension expressed as the median value d₅₀ of the particles size distribution which is preferably in a range of more than 5 to 50 μm. More preferably the d50 is in a range of 6 to 35 μm; most preferably in a range of 7 to 30 μm and further more preferably in a range of 8 to 25 μm.

Effect pigments consisting of the colored hectorites without further coatings can be applied to substrates by various techniques. They exhibit a strong color effect. When seen under a light source of polarized light they exhibit a polarization effect which is deemed to be caused by the ordered structure of intercalated dye molecules. This polarization effect enables an observer to perceive a different optical impression such as color strength when observing the application of the effect pigments under different angles of incidence and/or observance (pleochroism). It is especially observable when the incident light source is plane polarized light. For example, when applied on a black substrate and observed under a low angle of perception the absorption color of the dye is seen, whereas near the angle of glance a different color can be seen.

Below a d₅₀ of 5 μm these optical effects are not visible or are hardly be visible. Above a d₅₀ of 50 μm the particles become too large and tend to be misaligned when applied on a substrate.

The particles size distribution and thus also the d₅₀-value is determined by laser scattering. The measurements are conducted with a Horiba LA950 (Retsch Technology, Germany) laser scattering instrument applying a refractive index of 1.59. The results are determined as volume averaged particles size distribution based on equivalent spheres. The d₅₀-value (median) denotes to the value where 50% of the volume averaged size distribution is below and 50% are above this value.

Effect pigment according to any of the foregoing claims, wherein the average thickness h₅₀ of the clay is preferably in a range of 5 to 500 nm, wherein the thickness distribution is determined by SEM on cross-sections of coated effect pigments. About 100 particles should be measured. Preferred ranges of h₅₀ are 10 to 300 nm, further preferred 13 to 200 nm, more preferred 15 to 100 nm and most preferred 17 to 40 nm.

The standard deviation of the thickness distribution is rather small and is in a range of 15 to 50 nm, preferably in a range of 20 to 35 nm.

The relative standard deviation (standard deviation divided by the mean thickness) is in a range of 40% to 90% and preferably in a range of 50 to 80%.

5

Based on the d₅₀- and h₅₀ values an average aspect ratio can be defined to be d₅₀/h₅₀. The colored hectorites of this invention preferably have aspect ratios in a range of 10-10,000, further preferably aspect ratios in a range of 100-5,000, more preferably in a range of 300 to 3,000 and most preferably in a range of 400 to 2,000 and further most preferably in a n range of 410 to 1,000.

A striking difference of the colored hectorites of this invention from, for example, known colored montmorillonites lies the fact that due to their high exchange rate and especially due to their large sizes the hectorites have a significant higher number of dye molecules intercalated than in case of montmorillonites.

This can be characterized by introducing the parameter N_{DP} which is a measure of the average number of dye molecules per colored hectorite particle. N_{DP} is calculated by the following simplified formula:

N_{DP}=10⁶×CE_{clay}×n_{uc}/A_{uc} (III)

Herein is CE_{clay} the area of equivalent circles of the hectorite platelets assumed to have a disc form. Dye molecules can adsorb on both sides of the disc area. For convenience this area is calculated this from the d₅₀-values give in μm obtained from the laser scattering measurements mentioned above. Thus it is:

N_{DP}=2×10⁶ π(d₅₀/2)²×n_{uc}/A_{uc} (IV)

A_{uc} is the unit cell area which is for the sake of simplicity supposed to be 0.5 nm² throughout this invention. n_{uc} is the number of monovalent cations per unit cell area which is typically 1 for the hectorites used as layered silicate material in the present invention and 0.7, for example, for a typical montmorillonite.

This parameter can be seen as a measure of the number of dye molecules in the composite particles. The dye molecules have on one hand a strong lateral order due to their intercalation in the hectorite. On the other hand, when the colored hectorite is applied to a surface, due to their platelet-form the hectorites tend to align themselves in a plan-parallel manner to the surface plane they are applied on. These effects result in a high spatial orientation of the intercalated dye molecules.

The parameter N_{DP} is preferably in a range of 3.5×10⁸ to 1.5×10¹⁰, more preferably in a range of 4×10⁸ to 1.5×10¹⁰, further more preferably in a range of 4.1×10⁸ to 5×10⁹, even further more preferably in a range of 4.2×10⁸ to 1.5×10⁹ and most preferably in a range of 4.3×10⁸ to 1×10⁹

In contrast, N_{DP} is about more than one to three order of magnitudes lower for colored montmorillonites.

The cationic dye used for the intercalation can be preferably selected from the dye classes of azo, azamethylene, azine, anthrachinone, acridine, oxazine, polymethine, thiazine, triarylmethane, colored metal complexes or mixtures thereof.

A preferred group are triarylmethane dyes. A generic formula of this class of dyes can be represented by the mesomeric structure of formula (V):

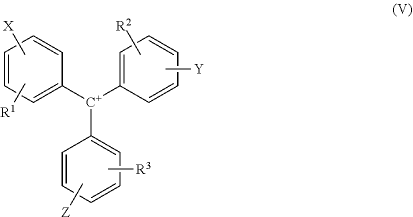

Herein R₁, R₂ and R₃ are independently H or CH₃ and are located at the ortho, meta or para position with respect to the C-atom bonded to the central carbenium ion with preference given to the meta position. X and Y are independently NR₄R₆ or OH, Z is H or NR₄R₅, wherein R₄ is independently H, CH₃ or C₂H₅ and R₅, R₆ are independently H, CH₃ or C₆H₅ with the proviso that at least one of R₄, R₅ or R₆ are H. X, Y and Z can be independently bonded in the meta or para position with respect to the central carbenium ion, with the para position being preferred.

Typical examples for triphenylmethane dyes are malachite green, brilliant green, methyl violet, fuchsine, aniline red, crystal violet, methyl green, aniline blue or victoria blue.

A further preferred group are acrydine or (thio)xanthene dyes which can be represented by the mesomeric structure according to formula (VI):

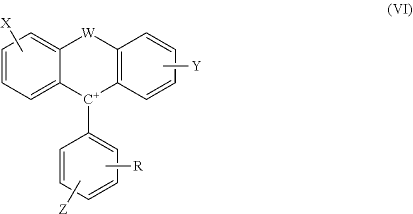

Herein W is NH for acrydine, O for xanthene and S for thioxanthene dyes. R is independently H, CH₃, C₂H₅, COOH or phenyl. X, Y and Z have the meaning described above for formula (V).

As thiazine dyes it is preferred to use phenothiazine dyes and derivates thereof. Preferred examples of phenothiazine derivates are methylene blue, methylene green or safranine red.

From the group of azo dyes a preferred dye is red 46.

From the group of polymethine dyes especially preferred are cyanine dyes. A specific examples of this class is Astra Yellow G.

In order to achieve an appealing optical effect the dyes are preferably chosen in such way that they exhibit a strong color effect. In preferred embodiments the cationic dyes solubilized in a common solvent exhibit therefore an absorption spectra which has a maximum of absorption bands in the visible wavelength range from above 450 nm to 750 nm. More preferably the maximum of the absorption bands is located in a wavelength range of 460 nm to 740 nm and most preferably in a wavelength range of 470 to 730 nm.

Furthermore, it is preferred that the dye has only on absorption band in the visible range.

Dyes which have the maximum of an absorption band outside the visible wavelength range are colored if part of an absorption band extends into the visible wavelength range. Such dyes usually do not have a strong and clear color and are therefore not preferred.

In preferred embodiments the effect pigments according to this invention do not use certain cationic dyes. Such preferably excluded cationic dyes are based on either [Ru(bipy)₃]²⁺ or N-hexadecyl-4-(3,4,5-trimethoxystyryl)-pyridinium, [Cu(trien)]²⁺, [Cu(dppb)₂]²⁺ or derivatives thereof. In [Cu(dppb)₂]²⁺ dppb means 1,2-bis(diphenylphosphino)-benzene. [Cu(trien)]²⁺ and [Cu(dppb)₂]²⁺ are rather small in size and therefore their hectorite intercalates are more susceptible to acid attack.

[Ru(bipy)₃]²⁺ or N-hexadecyl-4-(3,4,5-trimethoxystyryl)-pyridinium, exhibit an absorption maximum outside the preferred visible range and do not lead to an aesthetic color. Furthermore, the acid stability of all these dyes was mood.

Effect Pigments Based on Substrates of Colored Hectorites:

In a further embodiment of this invention the above described colored hectorites are used as substrate for the preparation of further effect pigments. This effect pigment comprises as a substrate a colored hectorite as described above and a layer thereon comprising at least one layer with a high index of refraction >1.8 or a semitransparent metal.

In preferred embodiments the at least one layer with a high index of refraction >1.8 comprises a metal oxide which is selected from the group consisting of TiO₂ (rutil), TiO₂ (anatase), Fe₂O₃, ZrO₂, SnO₂, ZnO, TiFe₂O₅, Fe₃O, TiFe₂O₅, FeTiO₃, BiOCl, CoO, C0₃O₄, Cr₂O₃, VO₂, V₂O₃, Sn(Sb)O₂, iron titanates, iron oxide hydrates, titanium suboxides (reduced titanium species having oxidation states from <4 to 2) bismuth vanadate, cobalt aluminate and mixtures or mixed phases of these compounds with one another or with other metal oxides.

In another embodiment of the invention the layer with a high index of refraction >1.8 comprises a metal sulfide which is selected from the group consisting of sulfides of tin, silver, lanthanum, rare earth metals, preferably cerium, chromium, molybdenum, tungsten, iron, cobalt and/or nickel and mixtures or mixed phases of these compounds with one another or with other metal sulfides.

Generally the layer thickness ranges from 10 to 1000 nm, preferably from 30 to 300 nm.

In another embodiment of the invention the effect pigment comprises said at least one layer with a high index of refraction >1.8 comprises a semitransparent metal which is selected from the group consisting of chromium, silver, aluminum, copper, gold, tin, titanium, molybdenum, tungsten, iron, cobalt and/or nickel and mixtures or mixed phases of these compounds with one another.

The semi-transparent metal layer has typically a thickness of between 5 and 30 nm, especially between 7 and 20 nm.

The metal layer can be obtained by wet chemical coating or by chemical vapor deposition, for example, gas phase deposition of metal carbonyls. The substrate is suspended in an aqueous and/or organic solvent containing medium in the presence of a metal compound and is deposited onto the substrate by addition of a reducing agent. The metal compound is, for example, silver nitrate or nickel acetyl acetonate (WO 2003/37993).

According to EP-A-353544 the following compounds can be used as reducing agents for the wet chemical coating: aldehydes (formaldehyde, acetaldehyde, benzalaldehyde), ketones (acetone), carbonic acids and salts thereof (tartaric acid, ascorbinic acid), reductones (isoascorbinic acid, triosereductone, reductine acid), and reducing sugars (glucose). However, it is also possible to use reducing alcohols (allyl alcohol), polyols and polyphenols, sulfites, hydrogensulfites, dithionites, hypophosphites, hydrazine, boron nitrogen compounds, metal hydrides and complex hydrides of aluminum and boron. The deposition of the metal layer can furthermore be carried out with the aid of a CVD method. Methods of this type are known. Fluidised-bed reactors are preferably employed for this purpose. EP-A-0741 170 describes the deposition of aluminum layers by reduction of alkylaluminum compounds using hydrocarbons in a stream of inert gas. The metal layers can furthermore be deposited by gas-phase decomposition of the corresponding metal carbonyls in a heatable fluidised-bed reactor, as described in EP-A-045851. Further details on this method are given in WO 1993/12182. A further process for the deposition of thin metal layers, which can be used in the present case for the application of the metal layer to the substrate, is the known method for vapor deposition of metals in a high vacuum. It is described in detail in Vakuum-Beschichtung [Vacuum Coating], Volumes 1-5; Editors Frey, Kienel and Lobl, VDI-Verlag, 1995. In the sputtering process, a gas discharge (plasma) is ignited between the support and the coating material, which is in the form of plates (target). The coating material is bombarded with high-energy ions from the plasma, for example argon ions, and thus removed or atomised. The atoms or molecules of the atomised coating material are precipitated on the support and form the desired thin layer. The sputtering process is described in Vakuum-Beschichtung [Vacuum Coating], Volumes 1-5; Editors Frey, Kienel and Lobl, VDI-Verlag, 1995.

In a further embodiment the substrate is first coated with an anti-bleeding layer, before coating with a high refractive index layer. Such anti-bleeding layer may prevent bleeding of dye molecules intercalated in the hectorite substrate or may prevent dissolving of any ions such as Mg²⁺, or Li⁺. The anti-bleeding layer is preferably selected from the group consisting from SiO₂, Al₂O₃, ZrO₂ or mixtures thereof. It is preferred to use such low-refractive index materials as this layer shall preferably not influence the optical properties of the effect pigment.

In an especially preferred embodiment, the interference pigments on the basis of the colored hectorite substrate comprise at least one multilayer coating having a stack of:

a) a layer with a high index of refraction >1.8, preferably >2.1,

b) a layer with a low index of refraction <1.8 and

c) a layer with a high index of refraction >1.8, preferably >2.1,

(d) optionally an outer protective layer.

Particularly suitable materials for layer a) or independently layer c) are metal oxides, metal sulfides, or metal oxide mixtures, such as TiO₂, Fe₂O₃, TiFe₂O₅, Fe₃O₄, BiOCl, CoO, Co₃O₄, Cr₂O₃, VO₂, V₂O₃, Sn(Sb)O₂, SnO₂, ZrO₂, iron titanates, iron oxide hydrates, titanium suboxides (reduced titanium species having oxidation states from 2 to <4), bismuth vanadate, cobalt aluminate, and also mixtures or mixed phases of these compounds with one another or with other metal oxides.

Metal sulfide coatings are preferably selected from sulfides of tin, silver, lanthanum, rare earth metals, preferably cerium, chromium, molybdenum, tungsten, iron, cobalt and/or nickel.

The layers a) or c) with a high index of refraction are preferably a metal oxide. It is possible for the metal oxide to be a single oxide or a mixture of oxides, with or without absorbing properties, for example.

Preferred high refractive index materials are TiO₂, ZrO₂, Fe₂O₃, Fe₃O₄, Cr₂O₃ or ZnO, with TiO₂ being especially preferred.

It is possible to obtain pigments that are more Intense in color and more transparent by applying, on top of the TiO₂ layer, a metal oxide of low refractive index, such as SiO₂, Al₂O₃, AlOOH, B₂O₃ or a mixture thereof, preferably SiO₂, and optionally applying a further TiO₂ layer on top of the later layer (EP-A-892832, EP-A-753545, WO 1993/08237, WO 1998/53011, WO 1998112266, WO 199838254, WO 1999/20695, WO 2000/42111 and EP-A-1213330). Nonlimiting examples of suitable low index coating materials that can be used include silicon dioxide (SiO₂), aluminum oxide (Al₂O₃), and metal fluorides such as magnesium fluoride (MgF₂), aluminum fluoride (AlF₃), cerium fluoride (CeF₃), lanthanum fluoride (LaF₃), sodium aluminum fluorides (e.g., Na₃AlF₆ or Na₅Al₃F₁₄), neodymium fluoride (NdF₃), samarium fluoride (SmF₃), barium fluoride (BaF₂), calcium fluoride (CaF₂), lithium fluoride (LiF), combinations thereof, or any other low index material having an index of refraction of about 1.8 or less.

In further embodiments organic monomers and polymers can be utilized as low index materials, including dienes or alkenes such as acrylates (e.g., methacrylate), polymers of perfluoroalkenes, polytetrafluoroethylene (TEFLON), polymers of fluorinated ethylene propylene (FEP), parylene, p-xylene, combinations thereof, and the like. Additionally, the foregoing materials include evaporated, condensed and cross-linked transparent acrylate layers, which may be deposited by methods described in U.S. Pat. No. 5,877,895, the disclosure of which is incorporated herein by reference.

Particularly suitable materials for layer b) are metal oxides or the corresponding oxide hydrates, such as SiO₂, Al₂O₃, AlOOH, B₂O₃ or a mixture thereof, most preferably SiO₂.

Accordingly, preferred interference pigments comprise besides high refractive index layers a) and c), preferably of metal oxide, in addition as a layer b) a metal oxide of low refractive index, wherein the difference of the refractive indices of high too low refractive index materials is at least 0.3.

In another particularly preferred embodiment the present invention relates to interference pigments containing at least three alternating layers of high and low refractive index, such as, for example, TiO₂/SiO₂/TiO₂, (SnO₂)TiO₂/SiO₂/TiO₂, TiO₂/SiO₂/TiO₂/SiO₂/TiO₂, Fe₂O₃/SiO₂/TiO₂ or TiO₂/SiO₂/Fe₂O₃.

The thickness of the individual layers of high and low refractive index on the base substrate is essential for the optical properties of the pigment. The thickness of the individual layers, especially metal oxide layers, depends on the field of use and the desired interference colors to be achieved and is generally 10 to 1000 nm, preferably 15 to 600 nm, in particular 20 to 200 nm.

The thickness of layer a) is 10 to 550 nm, preferably 15 to 350 nm and, in particular, 20 to 200 nm. The thickness of layer b) is 10 to 1,000 nm, preferably 20 to 800 nm and, in particular, 30 to 600 nm. The thickness of layer c) is 10 to 550 nm, preferably 15 to 350 nm and, in particular, 20 to 200 nm.

Interlayers of absorbing or nonabsorbing materials can be present between layers a), b), c) and d). The thickness of the interlayers is 1 to 50 nm, preferably 1 to 40 nm and, in particular, 1 to 30 nm. Such an interlayer can, for example, consist of SnO₂. It is possible to force the rutile structure to be formed by adding small amounts of SnO₂ (see, for example, WO 1993/08237).

In a further embodiment the effect pigments of this invention wherein after the coating providing optical appearance an outer coating providing weatherstability and/or UV-stability is provided. Preferably this outer protecting coating comprises one or more of metal oxides chosen from the group consisting of cerium-oxide, SiO₂, Al₂O₃, ZnO, SnO₂, ZrO₂ or mixtures thereof.

Preferably this outer coating is finished with an organic surface modifier to impart a bonding to organic binder material after the effect pigment has been applied in a lacquer or printing ink, for example. This organic surface modifier is composed of suitable organofunctional silanes, titanates, aluminates or zirconates.

In one further-preferred embodiment the organic surface modifier is composed of organofunctional silanes used comprise at least one sane furnished with at least one functional bond group.

A functional bond group here is a functional group which is able to enter into chemical interaction with the binder. This chemical interaction may be composed of a covalent bond, a hydrogen bond or an iconic interaction, and so on.

The functional bond groups comprise, for example, acrylate, methacrylate, vinyl, amino, cyanate, isocyanate, epoxy, hydroxyl, thiol, ureido and/or carboxyl groups.

The choice of suitable functional group depends on the chemical nature of the binder. It is preferred to choose a functional group which is chemically compatible with the functionalities of the binder, in order to allow effective attachment. In regard to weather-stable pearlescent pigments this quality is very important, since in this way a sufficiently strong adhesion is provided between pigment and cured binder. This can be tested, for example, in adhesion tests such as the cross-cut test with condensation exposure, in accordance with DIN 50 017. Passing such a test is a necessary condition for the use of weather-stable pearlescent pigments in an automotive finish.

Organofunctional silanes suitable as surface modifiers, with corresponding functional groups, are available commercially. By way of example they include many representatives of the products produced by Evonik Rheinfelden, Germany and sold under the trade name “Dynasylan®”, and the Silquest® silanes produced by Momentive Performance Materials or the GENOSIL® silanes produced by Wacker Chemie AG, Germany.

Examples of such silanes are 3-methacryloyloxypropyl-trimethoxysilane (Dynasylan MEMO, Silquest A-174NT), vinyltri(m)ethoxysilane (Dynasylan VTMO and VTEO, Silquest A-151 and A-171), 3-mercaptopropyltri(m)-ethoxysilane (Dynasylan MTMO or 3201; Silquest A-189), 3-glycidyloxypropyltrimethoxysilane (Dynasylan GLYMO, Silquest A-187), tris(3-trimethoxysilylpropyl)isocyanurate (Silquest Y-11597), gamma-mercaptopropyltri-methoxysilane (Silquest A-189), bis(3-triethoxysilyl-propyl) polysulfide (Silquest A-1289), bis(3-triethoxy-silyl) disulfide (Silquest A-1589), beta-(3,4-epoxy-cyclohexyl)ethyltrimethoxysilane (Silquest A-186), gamma-isocyanatopropyltrimethoxysilane (Silquest A-Link 35, Genosil GF40), (methacryloyloxymethyl)trimethoxysilane (Genosil XL 33) and (isocyanatomethyl)trimethoxysilane (Genosil XL 43).

In one preferred embodiment the organofunctional silane or sane mixture that modify the protective metal oxide layer comprises at least one amino-functional silane. The amino function is a functional group which is able to enter into chemical interactions with the majority of groups present in binders. This interaction may constitute a covalent bond, such as with isocyanate functions of the binder, for example, or hydrogen bonds such as with OH or COOH functions, or else ionic interactions. It is therefore very suitable for the purpose of chemically attaching the effect pigment to different kinds of binder,

For this purpose it is preferred to take the following compounds: aminopropyltrimethoxysilane (Dynasylan AMMO; Silquest A-1110), aminopropyltriethoxysilane (Dynasylan AMEO) or N-(2-aminoethyl)-3-aminopropyltrimethoxysilane (Dynasylan DAMO, Silquest A-1120) or N-(2-aminoethyl)-3-aminopropyltriethoxysilane, triamino-functional trimethoxysilane (Silquest A-1130), bis(gamma-trimethoxysilylpropyl)amine (Silquest A-1170), N-ethyl-gamma-aminoisobutyltrimethoxysilane (Silquest A-Link 15), N-phenyl-gamma-aminopropyltrimethoxysilane (Silquest Y-9669), 4-amino-3,3-dimethylbutyltrimethoxysilane (Silquest Y-11637), (N-cyclohexylaminomethyl)-triethoxysilane (Genosil XL 926), (N-phenylaminomethyl)trimethoxysilane (Genosil XL 973), and mixtures thereof.

Surprisingly, further advantageous performance properties have been obtained by means of an organic chemical surface modification of the SiO₂ layer which comprises at least one silane having at least one functional bond group and at least one silane without a functional bond group.

In this case, with particular preference, each silane having at least one functional bond group, as described above, is an aminosilane.

Preferably the outer coating and the silanes are composed of metal oxides as disclosed in EP 1682622 B1, EP 1727864 B1, EP 2691478 B1 or EP 2904052 B1.

Method of Manufacture of Colored Hectorite:

A further embodiment of this invention is a method of manufacture of an effect pigment consisting of colored hectorites as described above, comprising the following steps:

a) providing a hectorite according to formula (I)

b) dispersing said hectorite in an aqueous solution or water/acetonitrile or water/alcohol mixtures and optionally impacting of mechanical shear-forces until a highly swollen state is reached by osmotic swelling, wherein the separation of silicate layers d is in a range of above 10 nm to less than 1,000 nm,

c) ionic exchange of cations K with a cationic dye to a degree of 50-100% of the CEC, wherein optionally a cationic surface modifier is present, wherein the mole ratio of cationic surface modifier to dye is in a range of 0 to 3, and

d) optionally separating the colored hectorites obtained in step b) from the aqueous solution or optionally concentrating the colored hectorites obtained in step b) from the aqueous solution and/or optionally washing the effect pigment.

A preferred method of producing the hectorite of formula (I) comprises the following steps:
i) Preparation of K₂Li₂Si₆O₁₄ glass by first heating an appropriate amount of SiO₂.xH₂O (91.4% SiO₂) at a temperature above 800° C. Then the product is mixed with appropriate amounts of Li₂CO₃ and K₂CO₃, wherein K is selected from the group consisting of Li⁺, Na⁺, K⁺, NH₄⁺, Rb⁺, Cs⁺ or mixtures thereof and preferably K is selected from the group of Li⁺ and Na⁺ and most preferably K is Na⁺. This mixture is heated to at least 1,000° C., preferably under argon atmosphere. The obtained K₂Li₂Si₆O₁₄, preferably Na₂Li₂Si₆O₁₄ glass may be preferably crushed to particle diameters of a few mm, milled and sieved to obtain particles with diameters of less than 375 μm, preferably less than 250 μm.
ii) Synthesis of K_{0.5}Mg_{2.5}Li_{0.5}Si₄O₁₀F₂, preferably Na_{0.5}Mg_{2.5}Li_{0.5}Si₄O₁₀F₂: dry SiO₂, MgO and MgF₂ are produced by heating appropriate amounts of SiO₂.xH₂O (91.4% SiO₂) and Mg(OH)₂.MgCO₃ (42.5% MgO) to at least 800° C. and by separately heating appropriate amounts of MgF₂.xH₂O (85%) to at least 250° C. The glass obtained from step i) is mixed in appropriate amounts with these three materials and with KF, preferably NaF (99%) and heated under inert atmosphere to a temperature of at least 1,200° C.
iii) Hydrothermal treatment of K_{0.5}Mg_{2.5}Li_{0.5}Si₄O₁₀F₂, preferably Na_{0.5}Mg_{2.5}Li_{0.5}Si₄O₁₀F₂by first adding MgCl₂ to the glass obtained from step ii) and forming an aqueous suspension of this mixture. The mixture is equilibrated and washed until a conductivity of less than 100 μS/cm is obtained. A solid-liquid separation is made, for example, by sedimentation. Hydrothermal treatment was carried out at 10 wt-% (solid/water) at least 300° C., preferably at least 320° C. for at least 35 hours. The final product is dried within a time range of 8 to 24 hours in a temperature range of 60 to 100° C.

The ratios of all components are chosen with respect to the desired final composition.

Step b) of swelling the hectorite by osmotic swelling is the key step of this method. The osmotic swelling of hectorites is described, for example, in S. Rosenfeldt, M. Stöter, M. Schlenk, T. Martin, R. Q. Albuquerque, S. Förster and J. Breu, Langmuir 2016, 32, p. 10582-10588. The swelling can be conducted such that the interlayer distance of the layers dₛ, which can be determined with SAXS (small angel X-ray scattering), is in a range of above 0.5 to less than 1,000 nm. Preferably dₛ is in a range of 5 to less than 400 nm and more preferably in a range of 10 to 200 nm and even more preferably in a range of 20 to 150 nm. The interlayer distance dₛ depends mainly on the volume fraction of the dispersed hectorites (see FIG. 2d) from the cited publication). The osmotic swelling can be done in a region of the so called Gouy-Chapman regime, which corresponds to a dₛ of up to about 30 nm. It can be also done in the so-called screening regime, wherein the interlayer distance dₛ becomes larger than the Debye-length and is typically larger than 30 nm.

The main advantage of such high particle interlayer distances dₛ is that the kinetic constrains for the intercalation of rather bulky dye molecules are lost. Therefore, step c) can be conducted rather easily with high speed and efficiency.

Should the dye used for intercalation not be well soluble in water mixtures of water and organic solvents, such as H₂O/acetonitrile, H₂O/acetone or H₂O/alcohol mixtures can be used for the osmotic swelling as well as for step c). Especially preferred are H₂O/acetonitrile mixtures.

The concentration of the hectorites in the osmotic swelling step is in a range of 0.1 to 5 wt.-%, preferably in a range of 0.2 to 3 wt.-% in a range of 0.5 to 2 wt.-%. These rather low concentrations are needed in order to achieve the desired strongly swollen state of the hectorites.

The osmotic swelling can be accelerated by impacting mechanical forces, preferably shear forces on the suspended hectorites.

In case the cations K are, at least partially, alkylammonium salts, a further step is necessary to transfer the initial hectorite tactoides having a cation K form the group of alkali ions, preferably of Li⁺ or Na⁺ into a delaminated form. The alkylammonium salts help to delaminate the initial hectorites to a substantially quantitative extent. They are operated in a water/alcohol mixture.

The alkylammonium salts preferably have 2 to 8 carbon atoms in their alkyl chain and more preferably 3 to 6 carbon atoms.

The alcohol used for ion exchange of K for alkylammonium is preferably a monoalcohol with 1 to 4 carbon atoms. Most preferred is a water/ethanol mixture be used as solvent of osmotic swelling.

The initial hectorites are dispersed in such water/alcohol mixture and then a solution of alkylammonium salt is added.

The concentration of the alkylammonium salts in the solvent mixture of water and monoalcohol with 1 to 4 carbon atoms is preferably in a range of 0.5 to 100 mmol/L.

In step c) the cations K, preferably Na⁺, undergo an ionic exchange with a cationic dye. In a preferred embodiment a cationic surface modifier is present in the suspension of hectorite and dye. Such cationic surface modifier enhanced the dispersion stability of the colored hectorites decreasing the sedimentation and agglomeration tendency.

The degree of ionic exchange of the cations K, preferably of Na⁺, by dye molecules is in a range of 50-100% of the CEC, preferably in a range of 60 to 90% and most preferably in a range of 65 to 85% of the CEC. The degree of exchange is mainly dependent on the molar ratio of the cationic dye and of the surface modifier as surface modifier molecules may compete with the dye molecules for external adsorption sites of the hectorite.

The cationic surface modifier is preferably a cationic polymer or oligomer. This cationic polymer or oligomer preferably comprises ammonium ions. Preferred cationic polymers or oligomers are polyethylenimines (PEI), polyacrylamide (PAM), polydiallyidimethylammoniumchloride (PDADMAC), polyvinylamine (PVAm), dicyanediamideformaldehyde (DCG), polyamidoamine (PAMAM) or polyaminoamidedichlorohydrine (PAE).

Preferred cationic surface modifiers are polyethylenimines which can be modified or pure polyethylenimines. The modified polyethylenimines are preferred ethoxylated polyethylenimines (PEIE).

Examples for polyethylenimine polymers are chosen from the Lupasol® product group from BASF such as Lupasol G 20, Lupasol G 35, Lupasol G 100, Lupasol HF, Lupasol P or Lupasol PS.

The molar ratio of surface modifier to cationic dye is preferably in a range of 0.1 to 2.8, more preferably in a range of 0.4 to 2, further more preferably in a range of 0.5 to 1.5 and most preferably in a range of 0.6 to 1.

The molar amount of the cationic polymers serving as surface modifier here is always referred to the molar amount of the respective monomer unit.

Above a ratio of 2.8 the coloring of the hectorites by the dye is too weak. Below a ratio of 0.1, preferably of 0.4 the sedimentation and agglomeration of the colored hectorites was too strong.

In optional step d) separating the colored hectorites obtained in step b) from the aqueous solution or optionally concentrating the colored hectorites obtained in step b) from the aqueous solution and/or optionally washing the effect pigment.

The separation of the colored hectorites is preferably done by sedimentation, decantation, centrifugation or flotation techniques. Another separation technique is spray-drying. In this case, however, the collected effect particle powder should be redispersed very soon, preferably in an aqueous solution, as the delaminated hectorite particles have a high tendency to re-agglomerate due to their high specific surface.

After concentrating or separating the colored hectorite particles from the aqueous solution they may be washed once or several times to remove excess surface modifier and the cations K and possibly excess dye molecules by adding solvent, preferably water, and then again separate the particles from the solvent.

The separating step will preferably be conducted in such way that the colored hectorites still remain in a preferably aqueous dispersion in a concentration of below 20 wt.-%, preferably below 10 wt.-% and more preferably below 5 wt-% and most preferably below 2 wt.-%.

Method of Manufacture of Coated and Colored Hectorites:

A further embodiment of this invention is a method of manufacturing of an effect pigment based on colored hectorite, which is produced by ion exchange process of an initial hectorite with a cationic dye, wherein the initial hectorite can be represented by the formula

K_{z/z}[LiₓMg_{(3.0-(x+y)□y}Si₄O₁₀F₂] (I);

wherein n is the charge of K and z=x+2y with 0.2<z<0.8;

x=0-0.8; y=0-0.4;

K is a cation chosen from a first group consisting of Li⁺, Na⁺, K⁺, NH₄⁺, Rb⁺, Cs⁺, Mg²⁺, Ca²⁺, Sr²⁺, Ba²⁺ or mixtures thereof or from a second group consisting of alkylammonium salts with 2 to 8 C-atoms, wherein the alkyl can be branched or linear, or from a mixture of cations from the first and the second group and represent octahedral lattice sites. This method comprises the following steps:

a) a step of coating the colored hectorite with a high refractive index material, followed by

b) separating or concentrating the coated effect pigment from the solvent of the reaction media of step a),

c) optionally a drying step of the effect pigment of step a) and,

d) optionally classifying the effect pigment.

The high refractive index material layer is preferably a metal oxide layer. The metal oxide layer can be applied by CVD (chemical vapor deposition) or by wet chemical coating. The metal oxide layers can be obtained by decomposition of metal carbonyls in the presence of water vapor (relatively low molecular weight metal oxides such as magnetite) or in the presence of oxygen and, where appropriate, water vapor (e.g. nickel oxide and cobalt oxide). The metal oxide layers are especially applied by means of oxidative gaseous phase decomposition of metal carbonyls (e.g. iron pentacarbonyl, chromium hexacarbonyl; EP-A-45 851), by means of hydrolytic gaseous phase decomposition of metal alcoholates (e.g. titanium and zirconium tetra-n- and -iso-propanolate; DE-A-41 40 900) or of metal halides (e.g. titanium tetrachloride; EP-A-338 428), by means of oxidative decomposition of organyl fin compounds (especially alkyl fin compounds such as tetrabutyltin and tetramethyltin (DEA-44 03 678) or by means of the gaseous phase hydrolysis of organyl silicon compounds (especially di-tert-butoxyacetoxysilane) described in EP-A-668 329, it being possible for the coating operation to be carried out in a fluidised-bed reactor (EP-A-045 851 and EP-A-106 235).

Layers of oxides of the metals zirconium, titanium, iron and zinc, oxide hydrates of those metals, iron titanates, titanium suboxides or mixtures thereof are preferably applied by precipitation by a wet chemical method, it being possible, where appropriate, for the metal oxides to be reduced. In the case of the wet chemical coating, the wet chemical coating methods developed for the production of pearlescent pigments may be used; these are described, for example, in DE-A-14 67 468, DE-A-19 59 988, DEA-20 09 566, DE-A-22 14 545, DE-A-22 15 191 , DE-A-22 44 298, DE-A-23 13 331, DE-A-25 22 572, DE-A-31 37 808, DE-A-31 37 809, DE-A-31 51 343, DE-A-31 51 354, DE-A-31 51 355, DE-A-32 11 602 and DE-A-32 35 017, DE 195 99 88, WO 93/08237, WO 1998/53001 and WO 2003/6558.

The metal oxide of high refractive index is preferably TiO₂ and/or iron oxide, and the metal oxide of low refractive index is preferably SO₂. Layers of TiO₂ can be in the rutile or anastase modification, wherein the rutile modification is preferred. TiO₂ layers can also be reduced by known means, for example ammonia, hydrogen, hydrocarbon vapor or mixtures thereof, or metal powders, as described in EP-A-735, 1 14, DEA-3433657, DE-A-4125134, EP-A-332071, EP-A-707,050, WO 1993/19131 or WO 2006/131472.

For the purpose of coating, the substrate colored hectorite particles are suspended in water and one or more hydrolysable metal salts are added at a pH suitable for the hydrolysis, which is so selected that the metal oxides or metal oxide hydrates are precipitated directly onto the particles without subsidiary precipitation occurring. The pH is usually kept constant by simultaneously adding a base.

The pigments are then optionally classified, separated off, washed, dried and, where appropriate, calcined, it being possible to optimize the calcining temperature with respect to the coating in question. If desired, after individual coatings have been applied, the pigments can be separated off, dried and, where appropriate, calcined, and then again re-suspended for the purpose of precipitating further layers.

The temperature of drying can be in a range of 20 to less than 100° C., preferably

In a range of 20-70° C. and most preferably in a range of 20-50° C. Further preferred are drying techniques like freeze drying, spray-drying or vacuum drying. Vacuum drying can be made under static or dynamic conditions.

Instead of drying or additionally calcination can be utilized in order to remove excess water from the metal oxide layers. Calcination can be conducted under inert atmosphere. The temperature of calcination must be carefully chosen to avoid decomposition of the dye molecules. The temperature is preferably in a range of 100 to 900° C., preferably of 120 to 700°, more preferably in a range of 130 to 500° C. and further more preferably in a range of 140 to 400° C. and most preferably in a range of 150 to 300° C. The upper limit of the temperature is mainly limited by the temperature stability of the dye intercalated to the hectorite substrate.

Surprisingly rather high temperatures for calcination can be employed. Without being bound to a theory the inventors assume that by the intercalation the dye molecules are stabilized against thermal decomposition to a certain extent.

The meta oxide layers are also obtainable, for example, in analogy to a method described in DE 195 01 307 A1, by producing the metal oxide layer by controlled hydrolysis of one or more metal acid esters, where appropriate in the presence of an organic solvent and a basic catalyst, by means of a sol-gel process. Suitable basic catalysts are, for example, amines, such as triethylamine, ethylenediamine, tributylamine, dimethylethanolamine and methoxypropylamine. The organic solvent is a water-miscible organic solvent such as a C₄ alcohol, especially isopropanol.

Suitable metal acid esters are selected from alkyl and aryl alcoholates, carboxylates, and carboxyl-radical- or alkyl-radical- or aryl-radical-substituted alkyl alcoholates or carboxylates of vanadium, titanium, zirconium, silicon, aluminum and boron. The use of triisopropyl aluminate, tetraisopropyl titanate, tetraisopropyl zirconate, tetraethyl orthosilicate and triethyl borate is preferred. In addition, acetylacetonates and acetoacetylacetonates of the aforementioned metals may be used. Preferred examples of that type of metal acid ester are zirconium acetylacetonate, aluminum acetylacetonate, titanium acetylacetonate and diisobutyloleyl acetoacetylaluminate or diisopropyloleyl acetoacetylacetonate.

As a metal oxide having a high refractive index, titanium dioxide is preferably used, the method described in U.S. Pat. No. 3,553,001 being used, in accordance with an embodiment of the present invention, for application of the titanium dioxide layers.

An aqueous titanium salt solution is slowly added to a suspension of the material being coated, which suspension has been heated to about 50-100° C., especially 70-80° C., and a substantially constant pH value of about from 0.5 to 5, especially about from 1.2 to 2.5, is maintained by simultaneously metering in a base such as, for example, aqueous ammonia solution or aqueous alkali metal hydroxide solution. As soon as the desired layer thickness of precipitated TiO₂ has been achieved, the addition of titanium salt solution and base is stopped. Addition of a precursor for Al₂O₃ or MgO in the starting solutions is a way for improving the morphology of the TiO₂ layer.

This method, also referred to as the “titration method”, is distinguished by the fact that an excess of titanium salt is avoided. That is achieved by feeding in for hydrolysis, per unit time, only that amount which is necessary for even coating with the hydrated TiO₂ and which can be taken up per unit time by the available. surface of the particles being coated. In principle, the anatase form of TiO₂ forms on the surface of the starting pigment. By adding small amounts of SnO₂, however, it is possible to force the rutile structure to be formed. For example, as described in WO 1993/08237, tin dioxide can be deposited before titanium dioxide precipitation.

In an especially preferred embodiment of the present invention the colored hectorite flakes are mixed with distilled water in a closed reactor and heated at about 90° C. The pH is set to about 1.8 to 2.2 and a preparation comprising TiOCl₂, HCl, glycine and distilled water is added slowly while keeping the pH constant (1.8 to 2,2) by continuous addition of 1 M NaOH solution. Reference is made to European patent application PCT/EP20081051910. By adding an amino acid, such as glycine, during the deposition of the TiO₂ it is possible to improve the quality of the TiO₂ coating to be formed. Advantageously, a preparation comprising TiOCl₂, HCl, and glycine and distilled water is added to the substrate flakes in water. The TiO₂ can optionally be reduced by usual procedures: U.S. Pat. No. 4,948,631 (NH₃, 750-850° C.), WO 1993/19131 (H₂, >900° C.) or DE-A-19843014 (solid reduction agent, such as, for example, silicon, >600° C.).

Where appropriate, a SiO₂ (protective) layer can be applied on top of the titanium dioxide layer, for which the following method may be used: A soda waterglass solution is metered into a suspension of the material being coated, which suspension has been heated to about 50-100° C., especially 70-80° C. The pH is maintained at from 4 to 10, preferably from 6.5 to 8.5, by simultaneously adding 10% hydrochloric acid. After addition of the waterglass solution, stirring is carried out for 30 minutes.

It is possible to obtain pigments that are more intense in color and more transparent by applying, on top of the TiO₂ layer, a metal oxide of “low” refractive index, that is to say a refractive index smaller than about 1.65, such as SiO₂, Al₂O₃, AlOOH, B₂O₃ or a mixture thereof, preferably SiO₂, and applying a further Fe₂O₃ and/or TiO₂ layer on top of the latter layer. Such multi-coated interference pigments comprising a colored hectorite substrate and alternating metal oxide layers of with high and low refractive index can be prepared in analogy to the processes described in WO 1998/53011 and WO 1999/20695.

Use of Effect Pigments:

The effect pigments according to this invention can be in coatings, printing inks, powder coating, cosmetics or plastics.

For the purpose of pigmenting organic materials, the effect pigments according to the invention may be used singly. It is, however, also possible, in order to achieve different hues or color effects, to add any desired amounts of other color-imparting constituents, such as white, colored, black or effect pigments, to the high molecular weight organic substances in addition to the effect pigments according to the invention. When colored pigments are used in admixture with the effect pigments according to the invention, the total amount is preferably from 0.1 to 10% by weight, based on the high molecular weight organic material.

The pigmenting of high molecular weight organic substances with the pigments according to the invention is carried out, for example, by admixing such a pigment, where appropriate in the form of a masterbatch, with the substrates using roll mills or mixing or grinding apparatuses. The pigmented material is then brought into the desired final form using methods known per se, such as calendaring, compression moulding, extrusion, coating, pouring or injection moulding. Any additives customary in the plastics industry, such as plasticisers, fillers or stabilisers, can be added to the polymer, in customary amounts, before or after incorporation of the pigment. In particular, in order to produce non-rigid shaped articles or to reduce their brittleness, it is desirable to add plasticisers, for example esters of phosphoric acid, phthalic acid or sebacic acid, to the high molecular weight compounds prior to shaping.

For pigmenting coatings and printing inks, the high molecular weight organic materials and the effect pigments according to the invention, where appropriate together with customary additives such as, for example, fillers, other pigments, siccatives or plasticisers, are finely dispersed or dissolved in the same organic solvent or aqueous solvent mixture, it being possible for the individual components to be dissolved or dispersed separately or for a number of components to be dissolved or dispersed together, and only thereafter for all the components to be brought together.

Dispersing an effect pigment according to the invention in the high molecular weight organic material being pigmented, and processing a pigment composition according to the invention, are preferably carried out subject to conditions under which only relatively weak shear forces occur so that the effect pigment is not broken up into smaller portions.

Plastics comprising the pigment of the invention in amounts of 0.1 to 50% by weight, in particular 0.5 to 7% by weight. In the coating sector, the pigments of the invention are employed in amounts of 0.1 to 10% by weight. In the pigmentation of binder systems, for example for paints and printing inks for intaglio, offset or screen printing, the pigment is incorporated into the printing ink in amounts of 0.1 to 50% by weight, preferably 1 to 30% by weight and in particular 4 to 15% by weight.

The colorations obtained, for example in plastics, coatings or printing inks, especially in coatings or printing inks, more especially in coatings, may be distinguished by excellent properties, especially by extremely high saturation, outstanding fastness properties, high color purity and high goniochromaticity.

When the high molecular weight material being pigmented is a coating, it is especially a specialty coating, very especially an automotive finish.

The effect pigments according to the invention are also suitable for cosmetic applications such as making-up the lips or the skin and for coloring the hair or the nails. Preferably the cationic dye used to color the hectorite is chosen to be a cosmetically acceptable dye (1223/2009 EG).

The invention accordingly relates also to a cosmetic preparation or formulation comprising from 0.0001 to 90% by weight of a pigment, especially an effect pigment, according to the invention and from 10 to 99.9999% of a cosmetically suitable carrier material, based on the total weight of the cosmetic preparation or formulation.

Such cosmetic preparations or formulations are, for example, lipsticks, blushers, foundations, nail varnishes and haft shampoos.

The pigments may be used singly or in the form of mixtures. It is, in addition, possible to use pigments according to the invention together with other pigments and/or colorants, for example in combinations as described hereinbefore or as known in cosmetic preparations.

The cosmetic preparations and formulations according to the invention preferably contain the pigment according to the invention in an amount from 0.005 to 50% by weight, based on the total weight of the preparation.

Suitable, carder materials for the cosmetic preparations and formulations according to the invention include the customary materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, blushers, eye-shadows, foundations, eyeliners, powder or nail varnishes.

If the preparations are in the form of stick's, for example lipsticks, eye-shadows, blushers or foundations, the preparations consist for a considerable part of fatty components, which may consist of one or more waxes, for example ozokerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or fatty esters thereof that are solid at 25° C., silicone waxes, such as methyloctadecane-oxypolysiloxane and poly(dimethylsiloxy)stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxystearates of calcium, magnesium, zirconium and aluminum.

The fatty component may also consist of a mixture of at least one wax and at least one oil, in which case the following oils, for example, are suitable: paraffin oil, purcelline oil, perhydrosqualene, sweet almond oil, avocado oil, calophyllum oil, castor oil, sesame oil, jojoba oil, mineral oils having a boiling point of about from 310 to 410° C., silicone oils, such as dimethylpolysiloxane, linoleyl alcohol, linolenyl alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyl dodecanol.

The fatty components in such preparations in the form of sticks may generally constitute up to 99.91% by weight of the total weight of the preparation.

The cosmetic preparations and formulations according to the invention may additionally comprise further constituents, such as, for example, glycols, polyethylene glycols, polypropylene glycols, monoalkanolamides, non-colored polymeric, inorganic or organic fillers, preservatives, UV filters or other adjuvants and additives customary in cosmetics, for example a natural or synthetic or partially synthetic di- or tri-glyceride, a mineral oil, a silicone oil, a wax, a fatty alcohol, a Guerbet alcohol or ester thereof, a lipophilic functional cosmetic active ingredient, including sun-protection filters, or a mixture; of such substances.

A lipophilic functional cosmetic active ingredient suitable for skin cosmetics, an active ingredient composition or an active ingredient extract is an ingredient or a mixture of ingredients that is approved for dermal or topical application. The following may be mentioned by way of example:

active ingredients having a cleansing action on the skin surface and the hair; these include all substances that serve to cleanse the skin, such as oils, soaps, synthetic detergents and solid substances; active ingredients having a deodorising and perspiration-inhibiting action: they include antiperspirants based on aluminum salts or zinc salts, deodorants comprising bactericidal or bacteriostatic deodorising substances, for example triclosan, hexachlorophene, alcohols and cationic substances, such as, for example, quaternary ammonium salts, and odour absorbers, for example Grillocin® (combination of zinc ricinoleate and various additives) or triethyl citrate (optionally in combination with an antioxidant, such as, for example, butyl hydroxyloluene) or ion-exchange resins; active ingredients that offer protection against sunlight (UV filters): suitable active ingredients are filter substances (sunscreens) that are able to absorb UV radiation from sunlight and convert it into heat; depending on the desired action, the following light-protection agents are preferred: light-protection agents that selectively absorb sunburn-causing high-energy UV radiation in the range of approximately from 280 to 315 nm (UV-B absorbers) and transmit the longer-wavelength range of, for example, from 315 to 400 nm (UV-A range), as we as light-protection agents that absorb only the longer-wavelength radiation of the UV-A range of from 315 to 400 nm (UV-A absorbers).

Suitable light-protection agents are, for example, organic UV absorbers from the Mass of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenyl-benzimidazolesulfonic acid and salts thereof, menthyl anthranilates, benzotriazole derivatives, and/or an inorganic micropigment selected from aluminum oxide- or silicon dioxide-coated TiO₂, zinc oxide or mica; active ingredients against insects (repellents) are agents that are intended to prevent insects from touching the skin and becoming active there; they drive insects away and evaporate slowly; the most frequently used repellent is diethyl toluamide (DEET); other common repellents will be found, for example, in “Pflegekosmetik” (W. Raab and U. Kindl, Gustav-Fischer-Verlag Stuttgart/New York, 1991) on page 161; active, ingredients for protection against chemical and mechanical influences: these include all substances that form a barrier between the skin and external harmful substances, such as, for example, paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers for protection against the effect of organic solvents, or substances based on mineral oils, vegetable oils or silicone oils as “lubricants” for protection against severe mechanical stresses on the skin; moisturising substances: the following substances, for example, are used as moisture-controlling agents (moisturisers): sodium lactate, urea, alcohols, sorbitol, glycerol, propylene glycol, collagen, elastin and hyaluronic acid; active ingredients having a keratoplastic effect: benzoyl peroxide, retinoic acid, colloidal sulfur and resorcinol; antimicrobial agents, such as, for example, triclosan or quaternary ammonium compounds; oily or oil-soluble vitamins or vitamin derivatives that can be applied dermally: for example vitamin A (retinol in the form of the free acid or derivatives thereof), panthenol, pantothenic acid, folic acid, and combinations thereof, vitamin E (tocapherol), vitamin F; essential fatty acids; or niacinamide (nicotinic acid amide); vitamin-based placenta extracts: active ingredient compositions comprising especially vitamins A, C, E. B-i , B₂, B₃, B₁₂, folic acid and biotin, amino acids and enzymes as well as compounds of the trace elements magnesium, silicon, phosphorus, calcium, manganese, iron or copper; skin repair complexes: obtainable from inactivated and disintegrated cultures of bacteria of the bifidus group; plants and plant extracts: for example arnica, aloe, beard lichen, ivy, stinging nettle, ginseng, henna, camomile, marigold, rosemary, sage, horsetail or thyme; animal extracts: for example royal jelly, propolis, proteins or thymus extracts; cosmetic oils that can be applied dermally: neutral oils of the Miglyol 812 type, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, rice-seed oil, castor oil, safflower oil, sesame oil, soybean oil, sunflower oil, tea tree oil, grapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is to say in the form of ointments or creams, they may likewise be anhydrous or aqueous. Such preparations and formulations are, for example, mascaras, eyeliners, foundations, blushers, eye-shadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional additives, such as, for example, perfumes, antioxidants, preservatives, gel-forming agents, UV filters, colorants, pigments, pearlescent agents, non-colored polymers as well as inorganic or organic fillers. If the preparations are in the form of a powder, they consist substantially of a mineral or inorganic or organic filler such as, for example, talcum, kaolin, starch, polyethylene powder or polyamide powder, as well as adjuvants such as binders, colorants etc.

Such preparations may likewise comprise various adjuvants conventionally employed in cosmetics, such as fragrances, antioxidants, preservatives etc.

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents.

In that embodiment, the colored polymer is present in an amount of approximately from 0.1 to 5% by weight.

The cosmetic preparations and formulations according to the invention may also be used for coloring the hair, in which case they are used in the form of shampoos, creams or gels that are composed of the base substances conventionality employed in the cosmetics industry and a pigment according to the invention.

The cosmetic preparations and formulations according to the invention are prepared in conventional manner, for example by mixing or stirring the components together, optionally with heating so that the mixtures melt.

Thus the present application envisions cosmetics, coatings, inks, paints, and plastic composition containing the effect pigment formed from a coated or uncoated colored hectorite.

### EXPERIMENTAL

A Preparation of Samples

### Preparation of Na_{0.5}Mg_{2.5}Li_{0.5}Si₄O₁₀F₂

Preparation of Na₂Li₂Si₆O₁₄ Glass

523.2 g of SiO₂.xH₂O (91.4% SiO₂, 478.2 g SiO₂) was heated in corundum crucible to 900° C. (heating time: 40 min, ramp: 3 K·min⁻¹). The product was mixed with 140.6 g of Na₂CO₃ (99%) and 98.0 g Li₂CO₃ (99%) and afterwards heated to 1100° C. (heating time: 1 hour) in a graphite crucible under argon atmosphere. The obtained Na₂Li₂Si₆O₁₄ glass was crushed (particle diameter: 1-2 mm), milled and sieved to a particle diameter <250 μm.

Na_{0.5}Mg_{2.5}Li_{0.5}Si₄O₁₀F₂ synthesis:

(i) A mixture of 454.9 g SiO₂.xH₂O (91.4% SiO₂, 415.8 g SiO₂, 2.560 eq), 390.9 g Mg(OH)₂MgCO₃ (42.5% MgO, 166.2 g MgO, 1.525 eq) was heated to 900° C. (heating time: 40 min, ramp: 3 K·min⁻¹). (ii) 177.3 g of MgF₂.xH₂O (85%, 150.7 g MgF₂, 0.895 eq) were heated to 275° C. (heating time: 24 hours, ramp 5 K·min⁻¹). (iii) 293.5 g of the Na₂Li₂Si₆O₁₄glass (0.240 eq) were mixed with the materials obtained from steps (i), (ii) and with NaF (99%, 23.8 g, 0.210 eq). The mixture was melted in an induction type furnace in a graphite crucible under argon atmosphere (heating time: 20 minutes, temperature: 1280° C.) and afterwards quenched by switching of the power supply.

Hydrothermal Treatment:

In a typical procedure 0.1 eq of MgCl₂(46% CEC) are added to Na_{0.5}Mg_{2.5}Li_{0.5}Si₄O₁₀F₂ synthetized in the previous step and were equilibrated for 24 hours. Afterwards the suspension is washed until a conductivity of less than 100 μS/cm is obtained. Solid-liquid separation was done by sedimentation. Hydrothermal treatment was carried out at 10 wt-% (solid/water) at 340° C. (heating ramp: 76.3 K·h⁻¹, dwell time: 48 hours, cooling ramp: 22.8 K·h⁻¹). The final product was dried within 12 hours at 80° C.

### Preparation of a Dye-PEIE-Modified Hectorite

### Examples 1 to 4

Modification of 100 g of Na_{0.5}Mg_{2.5}Li_{0.5}Si₄O₁₀F₂ (called Na_{0.5}-hectorite herein) was divided into ten parts, each containing about 10 g of Na_{0.5}-hectorite. In the first step Na_{0.5}-hectorite suspension was prepared by suspending 10 g of Na_{0.5}-hectorite in 1 L of Milli-Q water (1 wt-% concentration, corresponding to a volume fraction of about 0.37 vol-%) In this suspension the Na_{0.5}-hectorite became highly swollen and adjacent silicate layers were uniformly separated to an average spacing d of about 100 nm., which can be estimated from FIG. 2d in S. Rosenfeldt, M. Stöter, M. Schlenk, T. Martin, R. Q. Albuquerque, S. Förster and J. Breu, Langmuir 2016, 32, p. 10582-10588. The suspension was afterwards placed in overhead shaker and mixed overnight at laboratory conditions.

The dye solution was prepared by dissolution of a certain amount of the dye (Tab.1) in 0.5 L of Milli-Q water. Afterwards an adequate volume (Tab.1) of ethoxylated polyethylenimine (PEIE, Lupasol G 20 from BASF SE; 80 wt % in water) has been added to the dye solution. The dye-PEIE solution was subsequently placed in an overhead shaker and mixed overnight under laboratory conditions. To induce shear force modification of the swollen hectorite was carried out with a Silent Crusher at 14,000 rpm. The Na_{0.5}-hectorite suspension was homogenized with a Silent Crusher for 3 minutes and for another 5 minutes after the dye-PEIE solution was added to the Na_{0.5}-hectorite suspension.

TABLE 1
Final suspension composition of 10 g samples
with 90% CEC:10% CEC dye:modificator ratio.

| | | Total | | | | |
|---|---|---|---|---|---|---|
| | Amount of | Volume | | | | Na_{0.5}- |
| | Na_{0.5}- | Milli-Q | | Amount | Volume | hectorite |
| | hectorite | water | | of dye | of PEIE | (wt-% of |
| Sample | (g) | (ml) | Dye | (g) | (ml) | suspension) |
| Example 1 | 10.06 | 1500 | Basic red | 3.096 | 81 | 0.63 |
| | | | 14 | | | |
| Example 2 | 10.03 | 1500 | Safranine | 2.849 | 81 | 0.63 |
| | | | O (Saf) | | | |
| Example 3 | 10.06 | 1500 | Malachite | 2.972 | 81 | 0.63 |
| | | | green | | | |
| | | | (MG) | | | |
| Example 4 | 9.94 | 1500 | Methylene | 2.575 | 80 | 0.62 |
| | | | blue (MB) | | | |

### Comparative Examples 1 to 8

Commercially available montmorillonites (PGV montmorillonite (Polymer Grade) from Nanocor, Arlington Heights, Ill. 60004, USA and SWY-1 montmorillonite from Source Clay Minerals Repository, MO 65211, USA) were colored with the dyes red 14, methylenblue, malachite green and Safranine-O according to the procedure described above. The details of the preparation parameters are dispatched in table 2. The CEC was determined by the [Cu(trien)]²⁺ method (according to L. Amman, F. Bergaya, G. Lagaly, Clay Miner. 2005, 40, 441-453) and was found to be 119 meq/100 g for the PGV montmorillonite and 71 meq/100 g for the SWY-1 montmorillonite.

All colored montmorillonites showed in PXRD a single peak (maximum position given in Table 3) indicating complete exchange of Na-ions with the dye cations.

### Comparative Example 9

Hectorite according to the Na_{0.5}Mg_{2.5}Li_{0.5}Si₄O₁₀F₂ preparation described above without any coloring by a dye.

### Comparative Example 10

(according to D. A: Kunz, M. Leitl, L. Schade, J. Schmid, B. Bojer, U. Schwarz, G. Ozin, H. Yersin and J. Breu, small 2015, No. 7, 792-796)

The Na-hectorite used in Examples 1 to 4 was treated with a solution of Ru(bipy)₃²⁺ salt as a dye. The PXRD showed a single peak at 17.8 Å indicating complete exchange of Na-ions with Ru(bipy)₃²⁺.

TABLE 2
Parameter or preparation of Comparative Examples
with substrates based on montmorillonites

| | Kind of | | Weight of | Volume | | |
|---|---|---|---|---|---|---|
| | mont- | | mont- | of dye | Concentration | Weight |
| | morillonite | Kind | morillonite | solution | dye solutions | of dye |
| Sample | [mg] | of dye | [mg] | [ml] | [mol/l] | [mg] |
| Comp. | PGV | Red14 | 303.1 | 17.17 | 0.0237 | 154.9 |
| Example 1 | | | | | | |
| Comp. | PGV | MB | 302.9 | 15.15 | 0.0244 | 118.2 |
| Example 2 | | | | | | |
| Comp. | PGV | Mal | 306.9 | 17.06 | 0.0241 | 150.3 |
| Example 3 | | | | | | |
| Comp. | PGV | Saf | 306.9 | 18.47 | 0.0222 | 143.7 |
| Example 4 | | | | | | |
| Comp. | SWY-1 | Red14 | 192.6 | 8.13 | 0.0237 | 73.3 |
| Example 5 | | | | | | |
| Comp. | SWY-1 | MB | 272.9 | 8.30 | 0.0244 | 64.8 |
| Example 6 | | | | | | |
| Comp. | SWY-1 | Mal | 292.3 | 8.96 | 0.0241 | 79 |
| Example 7 | | | | | | |
| Comp. | SWY-1 | Saf | 305.7 | 10.20 | 0.0222 | 79.3 |
| Example 8 | | | | | | |

B Characterization of Samples

Method A: Static Light Scattering (SLS)

Three drops of a 0.8 wt.-% dispersion of the pigment were dropped into the flow cell (type LA950) and homogenized by stirring. The measurements were made with a Horiba LA950 (Retsch Technology, Germany). The results were determined as volume averaged particles size distribution based on equivalent spheres. All measurements were repeated three times and the average of the d₅₀ was determined.

Method B: Powder X-ray Diffractometry (PXRD)

The clay samples were prepared as a 0.3 wt.-% dispersion and three drops thereof were dropped on a Menzel glass and slowly dried. The XRD diffractogramms were measured using a Bragg-Brentano diffractometer (PANalytical X'Pert Pro) with wavelength λ=1.54187 Å (Cu (Kα1) radiation filtered with a Ni-filter) and a X'Celerator Scientific RTMS detector.

Method C: SEM Determination of the Thickness of Colored Hectorites:

The sample was dispersed in the clear coat (Sikkens Autoclear HSR Anti Scratch) and applied on a foil. Cross sections were prepared and under the SEM the thickness of 100 particles was measured to construct a thickness distribution curve.

TABLE 3
Layer Spacings, median sizes and calculated NDp
values for various Examples and Comp. Examples

| | | Spacing | | | |
|---|---|---|---|---|---|
| | | clay | | | |
| | | (without | Spacing | d₅₀ [μm] | |
| | | dye) | clay with | (Clay | |
| | | obtained by | dye | colored with | |
| Sample | Dye | XRD [Å] | [Å] | dye) | N_{DP} × 10⁶ |
| Comp. | Red14 | 12.6 | 16.9 | 0.51 | 0.57 |
| Example 1 | | | | | |
| Comp. | MB | 12.6 | 18.4 | 0.54 | 0.64 |
| Example 2 | | | | | |
| Comp. | Mal | 12.6 | 20.1 | 0.55 | 0.67 |
| Example 3 | | | | | |
| Comp. | Saf | 12.6 | 20.0 | 0.57 | 0.71 |
| Example 4 | | | | | |
| Comp. | Red14 | 11.0 | 17.6 | 2.2 | 10.6 |
| Example 5 | | | | | |
| Comp. | MB | 11.0 | 15.6 | 2.8 | 17.2 |
| Example 6 | | | | | |
| Comp. | Mal | 11.0 | 18.2 | 4.0 | 35.2 |
| Example 7 | | | | | |
| Comp. | Saf | 11.0 | 15.9 | 2.6 | 14.9 |
| Example 8 | | | | | |
| Example 1 | Red 46 | 12.4 | 18.5 | 17 | 908 |
| Example 2 | Saf | 12.4 | 19.1 | 15 | 707 |
| Example 3 | Mal | 12.4 | 18.6 | 12 | 452 |
| Example 4 | MB | 12.4 | 18.3 | 13 | 531 |

The Comparative Examples 5 to 8 were made by a montmorillonite clay which was already known to produce relatively large particles. However, the results shown in Table 3 clearly demonstrate, that the inventive Examples have a much larger D₅₀-value and therefore also much higher N_{DP} values which were calculated from equation (IV). Per hectorite particle more than one order of dye molecules can be intercalated than with montmorrilonites.

TABLE 4
Results of Determination of thickness distribution function:

| | | | | | | | | relative |
|---|---|---|---|---|---|---|---|---|
| | | | | | | Aspect | standard | standard |
| | Intercaleted | h₁₀ | h₅₀ | h₉₀ | hₘₑₐₙ | ratio | deviation | deviation |
| Sample | dye | [nm] | [nm] | [nm] | [nm] | d₅₀/h₅₀ | h [nm] | h [%] |
| Example 2 | Saf | 16.8 | 30.5 | 59.0 | 37.4 | 492 | 26.4 | 71 |
| Example 4 | MB | 12.1 | 21.1 | 59.3 | 30.9 | 616 | 26.4 | 59.3 |

Both Examples prove that the median thickness h₅₀ or the mean thickness h mean is much lower than usual substrates of pearlescent pigments, which is in the order of 80 to 2,000 nm. The thickness distribution is in both cases rather small as is demonstrated in the characteristics of the standard deviation values.

C Properties of Samples:

Acid Stability Test and Cation Analysis with AAS:
a) In order to determine the total amount of releasable Mg²⁺ or Al³⁺-ions a defined amount of samples of a clay sample were placed in a Teflon crucible. To this sample 10 ml of HCL (30 wt.-%), 3 ml phosphoric acid (85 wt.-%), 3 ml nitric acid (65 wt.-%) and 7 ml fluoroboric acid (48 wt.-%) were added subsequently. A few minutes later 30 ml water and 13 ml phosphoric acid (85 wt.-%) were added. The sample was placed in a microwave device (High Performance Microwave mls 1200 mega, MLS GmbH) and the following program was conducted: 8 min at 200 W, 5 min at 0 W, 8 min at 300 W, 5 min at 0 W, 7 min at 600 W, 10 min 0 W). The solution was filtered into a 100 ml volumetric flask. Buffer solutions fitted to the cations (Na⁺, Mg²⁺) were added and the beaker was filled up to the mark. The concentration of leached cations was determined by AAS using a SpectrAA-100, Varian.
b) The acid stability test was conducted in HCl at pH=1 at 75° C. for 6 h in a similar volume as mentioned above. The results obtained are reported in Table 4 as percentage of acid-leached cations with respect to the total amount determined by method a).

TABLE 5
Results for acid leaching tests with AAS:

| | | | Al in | Mg in |
|---|---|---|---|---|
| | | | solution | solution |
| | | | (mol-[%] of | (mol-[%] of |
| Sample | Dye | weight [mg] | total Al) | total Mg) |
| Comp. | Red14 | 107.4 | 8.3 | 16.5 |
| Example 1 | | | | |
| Comp. | MB | 123.6 | 5.6 | 8.9 |
| Example 2 | | | | |
| Comp. | Mal | 259.8 | 4.2 | 14.2 |
| Example 3 | | | | |
| Comp. | Saf | 129.2 | 6.4 | 9.7 |
| Example 4 | | | | |
| Comp. | Red14 | 111.3 | — | 20.5 |
| Example 5 | | | | |
| Comp. | MB | 141.1 | — | 9.6 |
| Example 6 | | | | |
| Comp. | Mal | 119.3 | — | 6.9 |
| Example 7 | | | | |
| Comp. | Saf | 153.1 | — | 11.8 |
| Example 8 | | | | |
| Comp. | none | 80.3 | 0 | 23.6 |
| Example 9 | | | | |
| Comp. | Ru(bipy)₃²⁺ | 113.5 | 0 | 23 |
| Example 10 | | | | |
| Example 1 | Red 14 | 132.1 | 0 | 3.0 |
| Example 2 | Saf | 124.0 | 0 | 0.0 |
| Example 3 | Mal | 114.5 | 0 | 0.0 |
| Example 4 | MB | 109.9 | 0 | 0.1 |

It is well known that especially hectorites have a lower stability against acids than montmorillonites (see e.g. F. Bergaya, B. K. G. Theng and G. Lagaly, Handbook of Clay Science, Development of Clay Science, Vol. 1, 2006, Elsevier, Chapter 7.1 “Acid Activation of Clay Minerals” from P. Komadel and J. Madejová).

A completely surprising result is therefore the extreme acid stability of the hectorites after intercalation of dye molecules. The inventors do not have an explanation for these findings.

In contrast the coloring with Ru(bipy)₃²⁺ (Comp. Example 10) lead to an extremely unstable intercalated hectorite. Without being bound to any theory, the inventors believe that this molecule has an equivalent area which is too small to properly cover the clay surface and therefore, the clay surface is still accessible to the impact of acid attack.

D Preparation of Pearlescent Pigments

### Example 5

### Hectorite SiO₂+TiO₂

450 g Hectorite suspension of Example 1 (pigment concentration: 1.25 wt.-%) were heated in a reactor to 80° C., accompanied by stirring. The pH was adjusted with diluted hydrochloric acid or diluted alkaline dye (depending on the starting pH) to 7.5.

5.6 g water glass solution (27 wt.-% SiO₂), mixed with 20 g demineralized water was then introduced slowly into the suspension and the pH was kept constant at pH 7.5. The suspension was subsequently stirred for 2 h and then the pH was adjusted to pH 2.0. A solution of 125 ml TiCl₄ (100 g TiO₂/l demineralized water) and a 10 wt.-% aqueous earthy base solution were then metered into the suspension. After the coating has ended, subsequent stirring for 1 h and sedimentation were carried out in order to remove disruptive ions.

After separation the pigment was showing a silvery interference combined with a red absorption color.

### Examples 6 to 8

These examples were made as example 5, but using the colored hectorites of Examples 2 to 4 instead of Example 1.

In all cases the examples were resulting in a silvery glossy pigment with additional corresponding absorption color.

The thickness distribution of the substrate was determined for Example 6 (based on Example 2 as substrate) and for Example 8 (based on Example 4 as substrate) according to method C and the median thickness h₅₀ were determined, Example 6 was found to a h₅₀ of 31 nm and example 8 of 21 nm.

### Example 9

### Hectorite+TiO₂

450 g Hectorite suspension of example 4 were heated hi a reactor to 80° C., accompanied by stirring. The pH was adjusted with diluted hydrochloric acid or diluted alkaline lye (depending on the starting pH) to 2.2.

A solution of 100 ml TiCl₄(100 g TiO₂/l demineralized water) and a 10 wt.-% aqueous earthy base solution were then metered into the suspension. After the coating had ended, subsequent stirring for 1 h and sedimentation were carded out in order to remove disruptive ions,

The separated pigment was showing a silvery interference combined with a blue absorption color.

### Example 10

### Hectorite+TiO₂-Alcoholic

600 g Hectorite suspension of example 4 was transferred into an alcoholic phase by adding isopropyl alcohol and decantering two times.

To resulting suspension 80 g of Ti(IV) isopropoxide was added and the reactor heated up to 70° C. The suspension was subsequently stirred for 1 h and then a mixture of 20 g deionized water and 60 g Isopropanol was metered into the suspension, After 7 h additional agitation cooling and sedimentation were carded out.

The separated pigment was showing a silvery interference combined with a pinkish red absorption color.

1. Effect pigment comprising a colored hectorite produced by an ion exchange process of an initial hectorite with a cationic dye, wherein the initial hectorite is represented by formula (I):
K_{z/n}[LiₓMg_{(3.0-(x+y)□y}Si₄O₁₀F₂] (I),
wherein n is a charge of K, z=x+2y, 0.2<z<0.8,
x=0-0.8, and y=0-0.4;
K represents one or more of Li⁺, Na⁺, K⁺, NH₄⁺, Rb⁺, Cs⁺, Mg²⁺, Ca²⁺, Sr²⁺, Ba²⁺, and an alkylammonium salt having 2 to 8 carbon atoms, wherein an alkyl group of the alkylammonium salt is branched or linear, and □ represents not occupied octahedral lattice sites.

2. Effect pigment according to claim 1, wherein the initial hectorite is represented by the formula:
K_{z/n}[LiₓMg_{(3.0-(x+y)□y}Si₄O₁₀F₂]; (II)
wherein z=0.35 to less than 0.8, y=0-0.1, and x=0.35-0.65.

3. Effect pigment according to claim 1,
wherein K represents one or more of Li⁺, Na⁺, and an alkylammonium salt of one or more of ethylamine, n-propylamine, n-butylamine, sec-butylamine, tert-butylamine, n-pentylamine, tert-amylamine, n-hexylamine, sec-hexylamine, 2-ethyl-1hexylamine, n-heptylamine, 2-aminoheptane, n-octylamine and tert-octylamine.

4. Effect pigment according to claim 1, wherein a lateral dimension d₅₀ of the colored hectorite is in a range of more than 5 and up to 50 μm.

5. Effect pigment according to claim 1, wherein the average thickness h₅₀ of the colored hectorite is in a range of 5 to 500 nm, the thickness distribution of the colored hectorite being determined by SEM on cross-sections of oriented effect pigments in a coating.

6. Effect pigment according to claim 1, wherein the colored hectorite has an aspect ratio as defined by d₅₀/h₅₀ in a range of 10-10,000.

7. Effect pigment according to claim 1, wherein an average number of dye molecules per equivalent hectorite particle N_{DP} is in a range of 3.5×10⁸ to 1.5×10¹⁰.

8. Effect pigment according to claim 1, wherein the initial hectorite has a cationic exchange capacity (CEC) in a range of 80 to 213 mval/100 g.

9. Effect pigment according to claim 1, wherein the cationic dye includes one or more of an azo-based dye, an azamethylene-based dye, an azine-based dye, an anthrachinone-based dye, an acridine-based dye, an oxazine-based dye, a polymethine-based dye, a thiazine-based dye, a triarylmethane-based dye, and colored metal complexes thereof.

10. Effect pigment according to claim 1, wherein the cationic dye does not include any of [Ru(bipy)₃]²⁺, N-hexadecyl-4-(3,4,5-trimethoxystyryl)-pyridinium, [Cu(trien)]²⁺, Cu(dppp)₂]²⁺ or derivatives thereof.

11. Effect pigment according to claim 1, wherein the cationic dye in a solubilized state has an absorption spectrum with a maximum of absorption band in the range from above 450 nm and up to 800 nm.

12. Effect pigment according to claim 1, further comprising a coating on the colored hectorite, the colored hectorite forming a substrate, the coating comprising at least one layer high having an index of refraction greater than 1.8 or a semitransparent metal.

13. Effect pigment according to claim 12, wherein the at least one layer with a high index of refraction >1.8 comprises one or more of TiO₂ (rutil), TiO₂ (anatas), Fe₂O₃, ZrO₂, SnO₂, ZnO, TiFe₂O₅, Fe₃O₄, BiOCl, CoO, Co₃O₄, Cr₂O₃, VO₂, V₂O₃, Sn(Sb)O₂, an iron titanate, an iron oxide hydrate, a titanium suboxide having an oxidation state from 2 to less than 4, bismuth vanadate, and cobalt aluminate.

14. Effect pigment according to claim 12 wherein the at least one layer comprises the semitransparent metal, the semitransparent metal has an index of refraction greater than 1.8, and the semitransparent metal includes one or more of chromium, silver, aluminum, copper, gold, tin, titanium, molybdenum, tungsten, iron, cobalt and nickel.

15. Effect pigment according to claim 12, wherein the effect pigment is produced by applying an anti-bleeding layer to the substrate before applying the coating.

16. Effect pigment according to claim 12, wherein the coating comprises a stack of:
a) a layer having an index of refraction greater than 1.8,
b) a layer having an index of refraction less than 1.8, and
c) a layer having an index of refraction greater than 1.8.

17. Effect pigment according to claim 23, wherein the outer protective layer provides weatherstability and/or UV-stability and includes one or more of cerium-oxide, SiO₂, Al₂O₃, ZnO, and SnO₂.

18. Method of manufacture of an effect pigment, the method comprising:
providing an initial hectorite represented by formula (I):
K_{z/n}[LiₓMg_{(3.0-(x+y)□y}Si₄O₁₀F₂] (I),
wherein n is a charge of K, z=x+2y, 0.2<z<0.8, x=0-0.8, and y=0-0.4, K represents one or more of Li⁺, Na⁺, K⁺, NH₄⁺, Rb⁺, Cs⁺, Mg²⁺, Ca²⁺, Sr²⁺, Ba²⁺, and an alkylammonium salt having 2 to 8 carbon atoms, wherein an alkyl group of the alkylammonium salt is branched or linear, and □ represents an unoccupied octahedral lattice site;
dispersing the initial hectorite in an aqueous solution, a water/acetonitrile mixture, or a water/alcohol mixture, and
ionically exchanging K with a cationic dye to a degree of 50-100% of a cationic exchange capacity (CEC) of the initial hectorite.

19-20. (canceled)

21. Effect pigment according to claim 1, wherein the colored hectorite has an aspect ratio as defined by d₅₀/h₅₀ in a range of 400 to 2,000.

22. Effect pigment according to claim 1, wherein the initial hectorite has a cationic exchange capacity (CEC) in a range of 100 to 160 mval/100 g.

23. Effect pigment according to claim 12, further comprising an outer protective layer.

24. Effect pigment according to claim 15, wherein the anti-bleeding layer includes one or more of SiO₂, Al₂O₃, and ZrO₂.

25. Effect pigment according to claim 16, wherein the stack further comprises d) an outer protective layer.

26. Effect pigment according to claim 12, wherein the coating comprises a stack of:
a) a layer having an index of refraction greater than 2.1,
b) a layer having an index of refraction less than 1.8, and
c) a layer having an index of refraction greater than 2.1.

27. Effect pigment according to claim 26, wherein the stack further comprises d) an outer protective layer.

28. A coating comprising the effect pigment according to claim 1 and an organic material.

29. A printing ink comprising the effect pigment according to claim 1 and an organic material.

30. A composition comprising the effect pigment according to claim 1, the composition comprising a powder coating.

31. A cosmetic comprising the effect pigment according to claim 1 and a carrier material.

32. A plastic comprising the effect pigment according to claim 1 and a polymer.
